(19) United States
(12) Patent Application Publication
(10) Pub. No.: US 2008/0214911 A1
(43) Pub. Date: Sep. 4, 2008

(54) Method and Device for Determining Blood Components Using Ratiometric Absolute Pulse Spectroscopy

(76) Inventor: Klaus Forstner, Tamm (DE)

Correspondence Address: FRIEDRICH KUEFFNER, 317 MADISON AVENUE, SUITE 910, NEW YORK, NY, 10017, US

(21) Appl. No.: 10/556,652

(22) PCT Filed: Mar. 15, 2004
(86) PCT No.: PCT/DE04/00515

§ 371 Date: Nov. 16, 2006

(30) Foreign Application Priority Data
May 13, 2003 (DE) 103213384

(51) Int. Cl. A61B 5/00

(52) U.S. Cl. 600/315; 600/322

(57) ABSTRACT

The method and the device according to the invention are used for the non-invasive measurement of concentrations of blood components. At least one light source generates light using spectrophotometry and the light is guided to at least one photodetector through a tissue present at the location of application and supplied with pulsating blood. At least the measuring signal of the photodetector is guided to an evaluation unit. The light signals of a first, second, third to (n+1)^{st} wavelength are generated at subsequent pairwise times T1 and T2, T3 and T4 and T5 and T6 to Tₙ and Tₙ₊₁. The evaluation unit takes into consideration the received signals of the photodetector for all wavelengths according to a defined arithmetic pattern and determines a concentration of the blood component. The inventive device comprises at least three light sources that generate light of different wavelength in relation to each other. The evaluation unit is provided with an arithmetic unit for carrying out logarithms, divisions, multiplications, additions and subtractions. The inventive method is especially used for determining the total hemoglobin concentration C_{Hb} and for determining physiological substances that are iatrogenically applied to the area that is supplied with pulsating blood.

The invention concerns a method for measuring blood components, wherein, with the use of spectrophotometry, light from at least one light source is generated and passed through a perfused tissue located at an application site to at least one photoelectric transducer, and wherein at least one measuring signal of the photoelectric transducer is conducted to an evaluation unit.

The invention also concerns a device for measuring blood components, which has at least one light source, at least one photoelectric transducer, and at least one evaluation unit connected with the photoelectric transducer.

Methods and devices of this type are used in the field of medical technology to perform blood tests without having to withdraw blood from the patient. Devices of this type are typically applied to the fingers, toes, ears, or nose of a patient.

Blood components that are associated with hemoglobin and those that are not can be distinguished. Those components which are associated with hemoglobin are present mainly in the red blood cells and to a lesser extent are dissolved in the blood.

Hemoglobin derivatives can be distinguished as functional and dysfunctional constituents. Functional constituents are the oxyhemoglobin and deoxyhemoglobin fraction, while the dysfunctional hemoglobin fractions include primarily carboxyhemoglobin, methemoglobin, and sulfinethemoglobin.

In addition to these hemoglobin components, the blood contains a large number of other substances that are independent of hemoglobin. Some of these substances have both diagnostic and therapeutic significance. In this regard, native substances are distinguished from iatrogenically administered substances.

Native components are those which are physiologically present in the blood or are present in pathologically altered form. Iatrogenic substances are substances administered by the physician, e.g., dyes for marking certain clinical parameters.

A diagnostic method that employs spectrophotometry to test the pulsating blood compartment in biological tissues is known as pulse spectroscopy.

It is necessary to distinguish whether the method of pulse spectroscopy is used to determine relative fractions with respect to a reference substance or an absolute concentration. Accordingly, fractional or relative pulse spectroscopy is to be distinguished from absolute pulse spectroscopy.

A well-known example of relative pulse spectroscopy is the determination of arterial oxygen saturation by the method of pulse oximetry. This method measures the percentage of hemoglobin to which oxygen is bound. In this case, hemoglobin is the reference substance; however, it cannot be determined as an absolute concentration by relative pulse spectroscopy.

Relative pulse spectroscopy can also be used to determine, e.g., dysfunctional hemoglobin saturations, such as carbon monoxide saturation. This is described in various publications, including by the author himself.

Absolute pulse spectroscopy determines substance concentrations that are present within the pulsating arterial or venous blood compartment. These substances can be corpuscularly bound (i.e., they are components of blood cells) or they can be dissolved in the blood plasma.

In principle, the substances that can be determined are not necessarily substances associated with hemoglobin. They can be present independently of this molecule.

The literature describes various methods of absolute pulse spectroscopy. The object of the invention is the novel method of ratiometric absolute pulse spectroscopy.

Previously known methods and devices of absolute pulse spectroscopy are presently not yet sufficiently suited for the determination of blood substance concentrations with acceptable clinical measuring accuracy.

Therefore, the objective of the present invention is to specify a method of ratiometric absolute pulse spectroscopy that achieves a degree of measurement accuracy that is clinically acceptable for the determination of substance concentrations.

In accordance with the invention, this objective is achieved by generating light signals of a first wavelength at two successive times T₁ and T₂, generating light signals of a second wavelength at two successive times T₃ and T₄, generating light signals of a third wavelength at two successive times T₅ and T₆, and continuing this procedure for n pairs of times at n wavelengths. The times Tₙ . . . Tₙ₊₁ have a well-defined relationship with respect to time. Time differences between the times can be small and can be disregarded in the evaluation in individual cases. The incoming signals from the photoelectric transducer for all n wavelengths are considered by the evaluation unit according to a predetermined computational model to determine the concentration of a blood component.

A further objective of the invention is to specify a device of the aforementioned type that allows improved accuracy of measurement.

In accordance with the invention, this objective is achieved by using at least three light sources that generate different wavelengths from one another and by using an evaluation unit that has an arithmetic unit both for taking logarithms and for performing divisions, multiplications, additions, and subtractions.

Measurement of the light absorption at different wavelengths and at different times makes it possible to eliminate unknown parameters by suitable combination of the measured values with one another. This makes it unnecessary either to determine these parameters themselves, by possibly complicated means, or to be forced to accept measuring inaccuracies that result from the elimination of these parameters. Rather, this elimination provides a simple and extremely accurate method, and a device for performing this method can be produced relatively simply and thus inexpensively.

In a signal-processing step for eliminating unknown parameters, the evaluation unit considers a quotient of parameters derived from measuring signals (“ratiometric method”).

A transformation of a division to a subtraction when the logarithms are taken is utilized by considering a quotient of the logarithmized measured values.

A simple instrument design is assisted by using light-emitting semiconductor diodes to generate the light. These conventional light-emitting diodes (or laser diodes) can be used as photometric emission elements without additional spectral filtering.

It helps to keep the size of the measuring instrument small if the incoming signal is received by semiconductor photodiodes. These semiconductor photodiodes have varied spectral sensitivities.

The simple generation of light of different emission frequencies can be achieved by using at least three different light sources.

An example of a typical application is the determination of the concentration of total hemoglobin.

In particular, it is also intended that concentrations of components that are not associated with hemoglobin should be determined. This is true for both native and iatrogenically administered blood substances.

Another application is the determination of both bilirubin derivatives and the total concentration of bilirubin.

It is also possible to determine the concentration of myoglobin.

Furthermore, the method can be used to determine the concentrations and concentration kinetics of iatrogenically administered dyes.

Specific embodiments of the invention are shown schematically in the drawings.

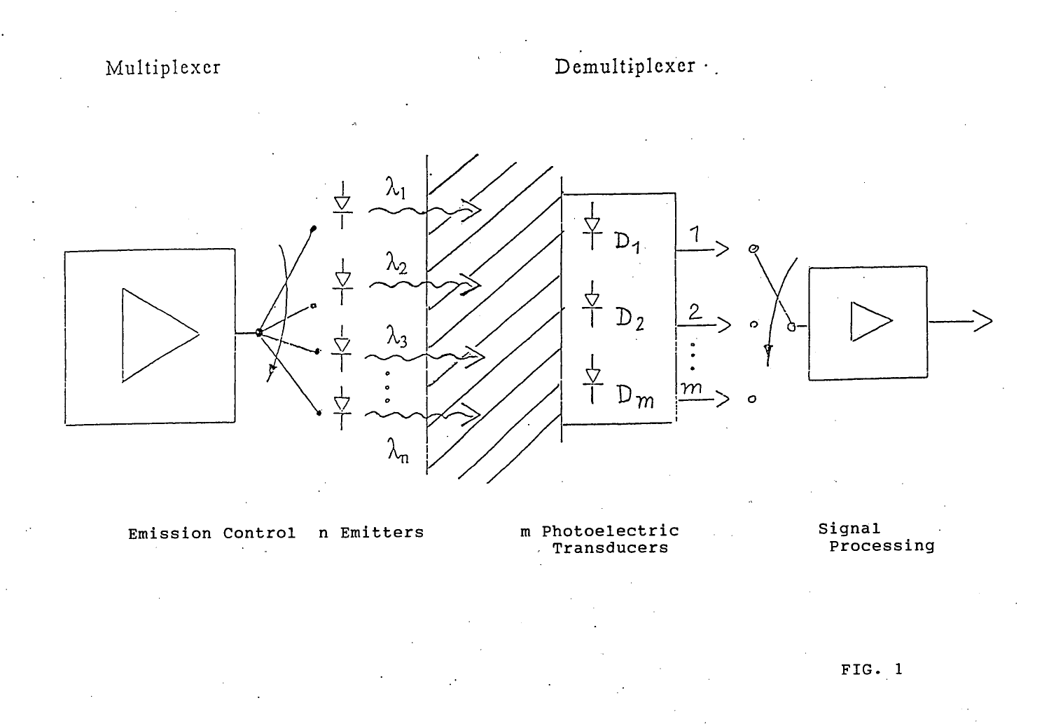

FIG. 1 shows the principle of the multiwavelength measuring technique in biological tissue.

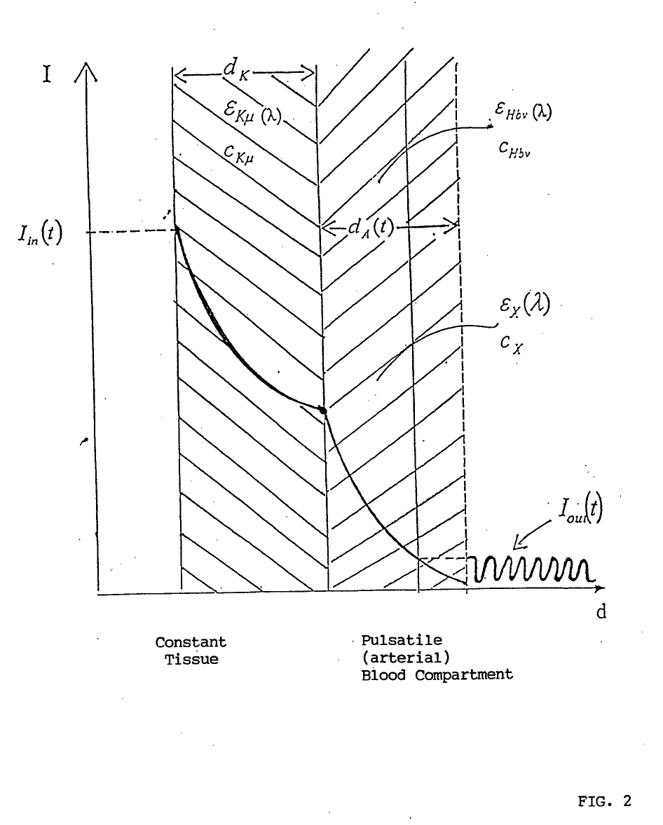

FIG. 2 shows the two-layer model of pulse spectroscopy with the inclusion of a pulsating non-Hb-associated absorber of concentration cₓ and of spectral absorption εₓ ( ).

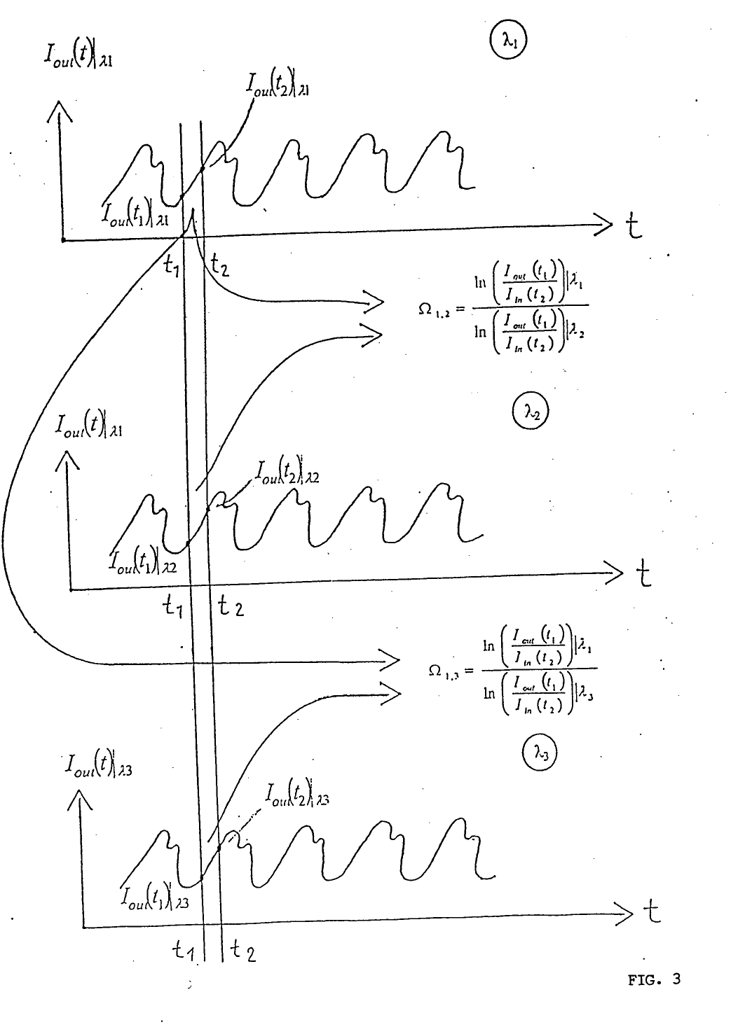

FIG. 3 shows a schematic representation of the formation of the measured-value variables Ω_{1,2} and Ω_{1,3} from two sampling times t₁ and t₂ in two plethysmograms of different wavelengths.

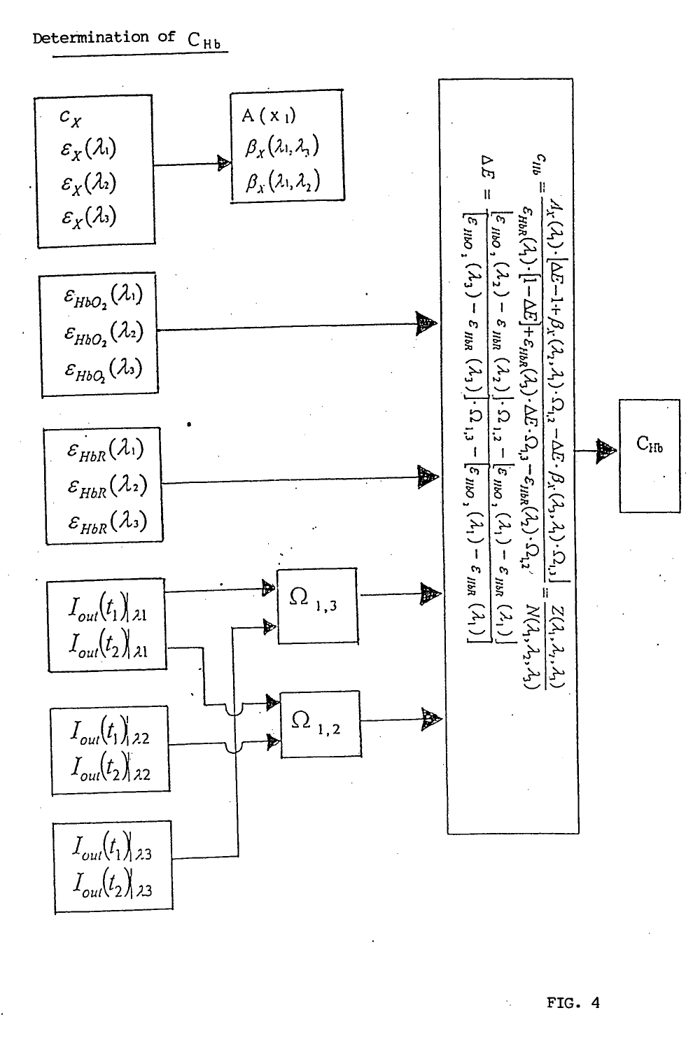

FIG. 4 shows a signal-flow diagram of the RAPS-method c_{Hb} with respect to the determination of the total hemoglobin concentration c_{Hb} with known pulsatile absorption of a substance X.

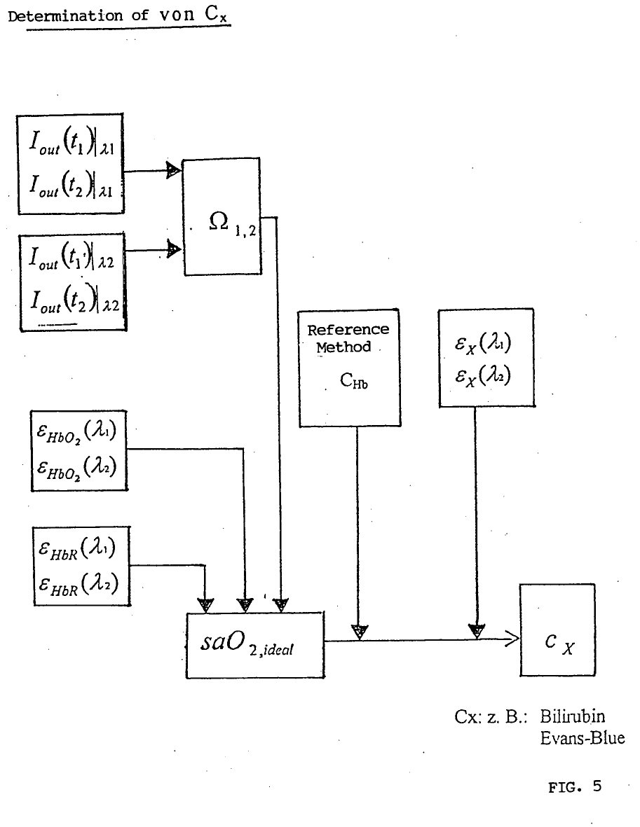

FIG. 5 shows a signal-flow diagram of the RAPS-method c_{Hb} with respect to the determination of the concentration cₓ of a pulsatile absorber with the spectral absorptions εₓ (₁) and εₓ (₂) (e.g., bilirubin, Evans blue) at known hemoglobin concentration c_{Hb}.

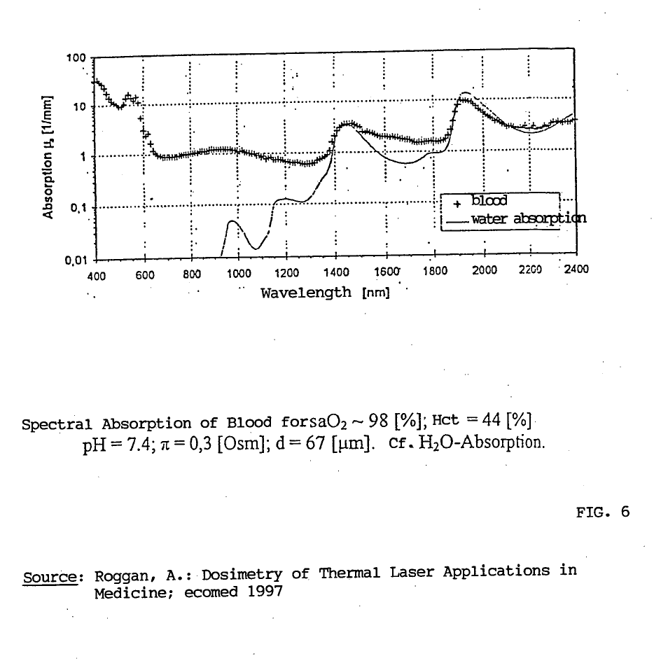

FIG. 6 shows the absorption spectrum of whole blood at an saO₂ of about 98% and the absorption spectrum of pure water. Please note the spectral absorption curve of H₂O between 1,000 (nm) and 1,600 (nm).

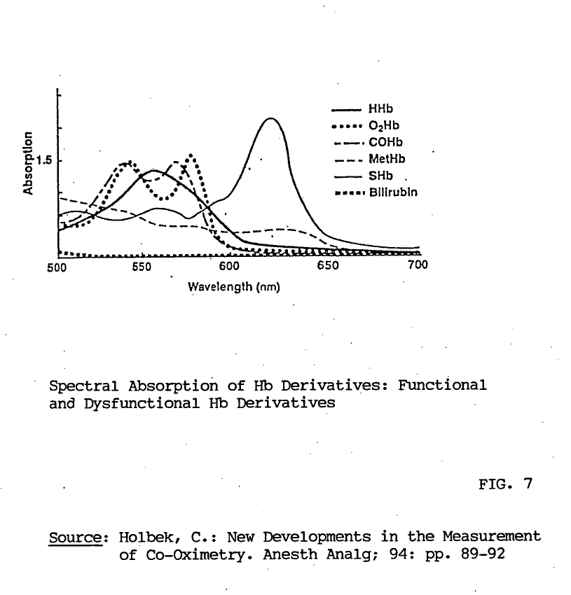

FIG. 7 shows VIS and NIR absorption spectra of functional and dysfunctional Hb derivatives.

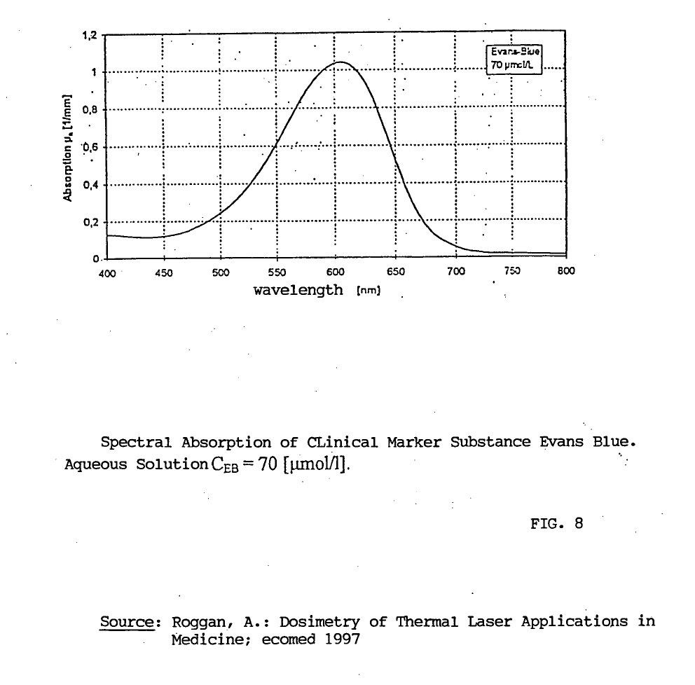

FIG. 8 shows the absorption spectrum of the clinical marker substance Evans blue (aqueous solution (μmoles/L)).

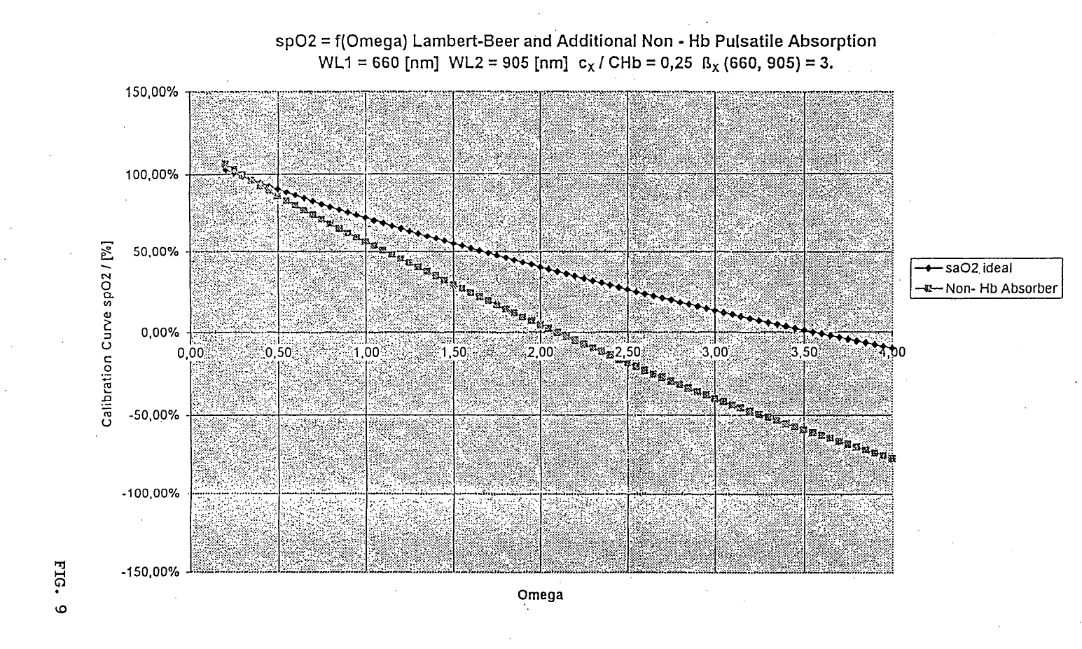

FIG. 9 shows the effect of a pulsatile non-Hb-associated absorber of concentration cₓ on the pulse oximetry calibration. Principle of the substance determination cₓ.

The device for measuring a concentration of blood components consists, for example, of three light sources (1, 2, 3) and a number of photoelectric transducers (4). The light sources (1, 2, 3) are realized in the form of light-emitting diodes. They are selected by a multiplexer and are connected to a control unit (5). The photoelectric transducers (4) are connected to an evaluation unit (6) via a light-phase-synchronous demultiplexer. The evaluation unit (6) has an arithmetic unit (7), which processes the measuring signals of the photoelectric transducer (4) according to preset computational instructions. The determined concentrations of the blood components can be displayed by a display (8) and/or can be passed along via an output device (9) or stored. The control unit (5) is connected with the evaluation unit (6) to allow functional coordination.

FIG. 2 shows a typical layer model for illustrating the principles of pulse spectroscopy. It shows the attenuation of the light intensity by the absorption in the nonpulsating part of the tissue (layer 1) and the attenuation within the pulsating part of the tissue (layer 2), which produces the pulsating fluctuation of the emerging light intensity.

In the arithmetic unit (7), the measured light intensities at different times and with respect to different wavelengths are computationally combined in such a way that certain unknown measuring parameters are eliminated. In the computational combination of the measured values, the transformation of a division to a subtraction by taking logarithms is utilized. Thus, if the quotient of two measured quantities at different times is formed, parameters that affect the given measured value but are constant with respect to time and are unnecessary are eliminated.

In detail, the computational steps explained below are carried out in the measured value processing. In this regard, it is assumed that the penetration of light can be approximately described by the Lambert-Beer law, and the attenuation of the light occurs primarily on the following biological substances:

1. On Hb derivatives;

2. On substances that are also present in the pulsating blood compartment but are not necessarily associated with Hb; and

3. On nonpulsating constant tissue.

The ratios are illustrated in FIG. 2.

The light penetration is given as follows for the signals I (FIG. 2):

$$\frac{{I}_{out} ( t )}{{I}_{i n} ( t )} = exp [ - {\sum }_{μ} {ɛ}_{K μ} \cdot {c}_{K μ} \cdot {d}_{K} ] \cdot exp [ - ( {\sum }_{v} {ɛ}_{Hbv} \cdot {c}_{Hbv} \cdot {d}_{A} ( t ) ) - {ɛ}_{X} \cdot {c}_{X} \cdot {d}_{A} ( t ) ] \qquad ( 1 )$$

ε are the underlying molar extinctions for the given substances, and c are the underlying concentrations. dₖ is the total thickness of the constant tissue. d_{A}(t) is the pulse-cyclical time-dependent thickness of the pulsating blood vessels.

If two times t₁ and t₂ are considered, the attenuation component in the constant tissue is eliminated:

$$\frac{{I}_{out} ( t )}{{I}_{i n} ( t )} = exp [ \begin{matrix}( ( {\sum }_{v} {ɛ}_{Hbv} \cdot {c}_{Hbv} ) + {ɛ}_{X} \cdot {c}_{X} ) + \\ [ {d}_{A} ( {t}_{2} ) - {d}_{A} ( {t}_{1} ) ]\end{matrix} ] \qquad ( 2 )$$

where

Δd_{A}=[d_{A}(t₀)−d_{A}(t₁)] (2a)

After taking the logarithm of both sides:

$$ln ( \frac{{I}_{out} ( {t}_{1} )}{{I}_{i n} ( {t}_{2} )} ) = [ {\sum }_{v} ( {ɛ}_{Hbv} \cdot {c}_{Hbv} ) + {ɛ}_{X} \cdot {c}_{X} ] \cdot Δ {d}_{A} \qquad ( 3 )$$

If O₂-Hb and HbR are used as examples of hemoglobin derivatives, the following is obtained:

$$\begin{matrix}\begin{matrix}{\sum }_{v = 1}^{2} ( {ɛ}_{Hbv} \cdot {c}_{Hbv} ) + {ɛ}_{X} \cdot {c}_{X} = {ɛ}_{{Hb O}_{2}} \cdot {c}_{{Hb O}_{2}} + \\ {ɛ}_{Hb R} \cdot {c}_{Hb R} + {ɛ}_{X} \cdot {c}_{X} \\ = [ \begin{matrix}( {ɛ}_{{Hb O}_{2}} - {ɛ}_{Hb R} ) \cdot {sa O}_{2} + \\ {ɛ}_{Hb R} + {ɛ}_{X} \cdot \frac{{c}_{X}}{{c}_{Hb}}\end{matrix} ] \cdot {c}_{Hb}\end{matrix} & ( 4 ) \\ where & \\ sa {O}_{2} = \frac{{c}_{Hb {O}_{2}}}{{c}_{Hb}} & ( 4 a ) \\ sa R = \frac{{c}_{Hb R}}{{c}_{Hb}} & ( 4 b ) \\ sa {O}_{2} + sa R = 1 & ( 4 c )\end{matrix}$$

This results in two distinct clinical applications of ratiometric absolute pulse spectroscopy. First, a blood component X can be continuously and noninvasively determined if the hemoglobin concentration c_{Hb} is known (measured, e.g., by reference method I). Second, the hemoglobin concentration c_{Hb} itself can be determined if the concentration of another pulsating absorber is known (II). In principle, both methods of determination can be realized independently of each other.

### I. Determination of the Concentration of a Substance X in the Pulsating Blood Compartment

As is well known, εₓ and cₓ are the molar extinction and the concentration of a blood component, which is not necessarily associated with hemoglobin.

If two wavelengths 1 and 2 are introduced into (4), then the following is obtained from (4) after division:

$$\begin{matrix}\frac{ln ( \frac{{I}_{out} ( {t}_{1} )}{{I}_{i n} ( {t}_{2} )} ) {λ}_{1}}{ln ( \frac{{I}_{out} ( {t}_{1} )}{{I}_{i n} ( {t}_{2} )} ) {λ}_{2}} = {Ω}_{1 , 2} \\ = \frac{\begin{matrix}[ {ɛ}_{Hb {O}_{2}} ( {λ}_{1} ) - {ɛ}_{Hb R} ( {λ}_{1} ) ] \cdot \\ sa {O}_{2} + {ɛ}_{Hb R} ( {λ}_{1} ) + {ɛ}_{X} ( {λ}_{1} ) \cdot \frac{{c}_{X}}{{c}_{Hb}}\end{matrix}}{\begin{matrix}[ {ɛ}_{Hb {O}_{2}} ( {λ}_{2} ) - {ɛ}_{Hb R} ( {λ}_{2} ) ] \cdot \\ sa {O}_{2} + {ɛ}_{Hb R} ( {λ}_{2} ) + {ɛ}_{X} ( {λ}_{2} ) \cdot \frac{{c}_{X}}{{c}_{Hb}}\end{matrix}}\end{matrix} \qquad ( 5 )$$

In this regard, Ω_{1,2} is the measured-value variable of the two wavelengths 1 and 2. The formation of the measured-value variables in the case of 3-wavelength pulse spectroscopy is shown in FIG. 3. After solving for saO₂, the following is obtained:

$$sa {O}_{2} = \frac{\begin{matrix}{ɛ}_{Hb R} ( {λ}_{1} ) - {ɛ}_{Hb R} ( {λ}_{2} ) \cdot {Ω}_{1 , 2} + \\ [ {ɛ}_{X} ( {λ}_{1} ) - {ɛ}_{X} ( {λ}_{2} ) \cdot {Ω}_{1 , 2} ] \cdot ( \frac{{c}_{X}}{{c}_{Hb}} )\end{matrix}}{\begin{matrix}{Ω}_{1 , 2} \cdot [ {ɛ}_{Hb {O}_{2}} ( {λ}_{2} ) - {ɛ}_{Hb R} ( {λ}_{2} ) ] - \\ [ {ɛ}_{Hb {O}_{2}} ( {λ}_{1} ) - {ɛ}_{Hb R} ( {λ}_{1} ) ]\end{matrix}} \qquad ( 6 )$$

In this regard, it is important to note that the modification of the determination of the arterial oxygen saturation is given by this equation if an absorbing substance X with the concentration cₓ and the extinctions ε_{x 2} and ε_{x 1} is present in the pulsating blood compartment. This can also be explicitly formulated:

$$sa {O}_{2} = sa {O}_{2 , ideal} + \frac{[ {ɛ}_{X} ( {λ}_{1} ) - {ɛ}_{X} ( {λ}_{2} ) \cdot {Ω}_{1 , 2} ] \cdot ( \frac{{c}_{X}}{{c}_{Hb}} )}{\begin{matrix}{Ω}_{1 , 2} \cdot [ {ɛ}_{Hb {O}_{2}} ( {λ}_{2} ) - {ɛ}_{Hb R} ( {λ}_{2} ) ] - \\ [ {ɛ}_{Hb {O}_{2}} ( {λ}_{1} ) - {ɛ}_{Hb R} ( {λ}_{1} ) ]\end{matrix}} \qquad ( 6 a )$$

Equations (6) and (6a) can be used to determine the concentration of substance X, which is not necessarily associated with hemoglobin. This substance can be, for example, bilirubin or other native blood substances, or this substance X can be an iatrogenically administered substance, e.g., a dye marker substance. These iatrogenically administered substances can also be administered to perform doping of native or pharmacologically active substances.

Finally, if the arterial oxygen saturation saO₂ is known (e.g., by pulse oximetry) and the hemoglobin concentration c_{Hb} is known (e.g., by reference method), cₓ can be determined, since all relevant molar extinctions and the measured-value variables Ω_{1,2} of the pulse spectroscopy are known.

As an example, FIG. 9 shows the effect of a concentration cₓ on the ideal pulse oximetry calibration (₁=660 (nm), ₂=905 (nm); cₓ/c_{Hb}=0.25, ₓ(660, 905)=3). It should be noted that for each value of the measured-value variables Ω_{1,2}, the assignment to the oxygen saturation varies in a characteristic way, depending on cₓ.

It should be pointed out that the aforesaid measured value variables Ω_{1,2} are not necessarily the measured-value variable that is the basis of the pulse oximetry determination of the arterial oxygen saturation.

### II. Determination of the Total Hemoglobin Concentration c_{Hb} when the Substance Concentration cₓ is Already Known

With the definitions of the spectral extinction ratio εₓ(₂/₁) and of the absorption Aₓ( )=εₓ( )cₓ, the following is obtained:

$$sa {O}_{2} = \frac{\begin{matrix}{ɛ}_{Hb R} ( {λ}_{1} ) - {ɛ}_{Hb R} ( {λ}_{2} ) \cdot {Ω}_{1 , 2} + \\ [ 1 - {β}_{X} ( {λ}_{2} , {λ}_{1} ) \cdot {Ω}_{1 , 2} ] \cdot ( \frac{{A}_{X} ( {λ}_{1} )}{{c}_{Hb}} )\end{matrix}}{\begin{matrix}{Ω}_{1 , 2} \cdot [ {ɛ}_{Hb {O}_{2}} ( {λ}_{2} ) - {ɛ}_{Hb R} ( {λ}_{2} ) ] - \\ [ {ɛ}_{Hb {O}_{2}} ( {λ}_{1} ) - {ɛ}_{Hb R} ( {λ}_{1} ) ]\end{matrix}} \qquad ( 7 )$$

This defining equation for saO₂ depends on the still unknown substance concentration c_{Hb} if Aₓ( ) and εₓ(₂/₁) are known. This concentration can then be determined by introducing another wavelength 3, and this is likewise used in accordance with the given system for the determination of saO₂.

The following is then obtained analogously to Equation (7) with the additional measurement variables Ω_{1,3}:

$$sa {O}_{2} = \frac{\begin{matrix}{ɛ}_{Hb R} ( {λ}_{1} ) - {ɛ}_{Hb R} ( {λ}_{3} ) \cdot {Ω}_{1 , 3} + \\ [ 1 - {β}_{X} ( {λ}_{3} , {λ}_{1} ) \cdot {Ω}_{1 , 3} ] \cdot ( \frac{{A}_{X} ( {λ}_{1} )}{{c}_{Hb}} )\end{matrix}}{\begin{matrix}{Ω}_{1 , 3} \cdot [ {ɛ}_{Hb {O}_{2}} ( {λ}_{3} ) - {ɛ}_{Hb R} ( {λ}_{3} ) ] - \\ [ {ɛ}_{Hb {O}_{2}} ( {λ}_{1} ) - {ɛ}_{Hb R} ( {λ}_{1} ) ]\end{matrix}} \qquad ( 8 )$$

The Equations (7) and (8) now represent two independent computational equations for the saturation saO₂. This can now be eliminated, and the unknown c_{Hb} can be determined. The following is then obtained:

$$\begin{matrix}\begin{matrix}{c}_{Hb} = \frac{{A}_{X} ( {λ}_{1} ) \cdot ⌊ \begin{matrix}Δ E - 1 + {β}_{X} ( {λ}_{2} , {λ}_{1} ) \cdot {Ω}_{1 , 2} - \\ Δ E \cdot {β}_{X} ( {λ}_{3} , {λ}_{1} ) \cdot {Ω}_{1 , 3}\end{matrix} ⌋}{\begin{matrix}{ɛ}_{Hb R} ( {λ}_{1} ) \cdot [ 1 - Δ E ] + {ɛ}_{Hb R} ( {λ}_{3} ) \cdot \\ Δ E \cdot {Ω}_{1 , 3} - {ɛ}_{Hb R} ( {λ}_{2} ) \cdot {Ω}_{1 , 2}\end{matrix}} \\ = \frac{Z ( {λ}_{1} , {λ}_{2} , {λ}_{3} )}{N ( {λ}_{1} , {λ}_{2} , {λ}_{3} )}\end{matrix} & ( 9 ) \\ Δ E = \frac{\begin{matrix}⌊ {ɛ}_{Hb {O}_{2}} ( {λ}_{2} ) - {ɛ}_{Hb R} ( {λ}_{2} ) ⌋ \cdot {Ω}_{1 , 2} - \\ ⌊ {ɛ}_{Hb {O}_{2}} ( {λ}_{1} ) - {ɛ}_{Hb R} ( {λ}_{1} ) ⌋\end{matrix}}{\begin{matrix}[ {ɛ}_{Hb {O}_{2}} ( {λ}_{3} ) - {ɛ}_{Hb R} ( {λ}_{3} ) ] \cdot {Ω}_{1 , 3} - \\ [ {ɛ}_{Hb {O}_{2}} ( {λ}_{1} ) - {ɛ}_{Hb R} ( {λ}_{1} ) ]\end{matrix}} & ( 9 a )\end{matrix}$$

In this regard, Aₓ(₃)=cₓεₓ (₁) is the known absorption of a substance X.

With the numerator term Z:

Z(λ₁,λ₂,λ₃)/A_{X}(λ₁)=[(ε_{HbO}_{₂}(λ₃)−ε_{HbR}(λ₃)·β_{X}(λ₂,λ₁)−(ε_{HbO}_{₂}(λ₂)·β_{X}(λ₃,λ₁)]·Ω_{1,2}·Ω_{1,3}+[(ε_{HbO}_{₂}(λ₂)−ε_{HbR}(λ₂)−(ε_{HbO}_{₂}(λ₁)−ε_{HbR}(λ₁)·β_{X}(λ₂,λ₁)]·Ω_{1,2}+[(ε_{HbO}_{₂}(λ₁)−ε_{HbR}(λ₁)·β_{X}(λ₃,λ₂)−(ε_{HbO}_{₂}(λ₃)−ε_{HbR}(λ₃)]·Ω_{1,3} (10)

And the denominator term N:

N(λ₁,λ₂,λ₃)=[(ε_{HbO}_{₂}(λ₂)−ε_{HbR}(λ₂)·ε_{HbR}(λ₃)−(ε_{HbO}_{₂}(λ₃)−ε_{HbR}(λ₃)·ε_{HbR}(λ₂)]·Ω_{1,2}·Ω_{1,3}+[(ε_{HbO}_{₂}(λ₁)−ε_{HbR}(λ₁)·ε_{HbR}(λ₂)−(ε_{HbO}_{₂}(λ₂)−ε_{HbR}(λ₂)·ε_{HbR}(λ₃)]·Ω_{1,2}+[(ε_{HbO}_{₂}(λ₃)—ε_{HbR}(λ₃)·ε_{HbR}(λ₂)−(ε_{HbO}_{₂}(λ₁)−(λ₁)·ε_{HbR}(λ₃)]·Ω_{1,3} (11)

The determination of the sought concentration c_{Hb} is therefore unique, because it is determined from the measured-value variables and the already known extinctions. The whole method that has been described is valid for the assumption of an idealized Lambert-Beer attenuation. This simplifying physical modeling can be adapted by empirically modified modeling to the real conditions or to the optical properties of the biological site of application.

If several dysfunctional Hb fractions are present, the total hemoglobin concentration is to be determined by the introduction of additional light wavelengths.

Ratiometric absolute pulse spectroscopy makes it possible to achieve the following results:

(1) The determination of the concentration of hemoglobin c_{Hb} and its derivatives if the concentration cₓ of an iatrogenically administered or native absorber X is present, which is not necessarily associated with Hb; and

(2) The determination of the concentration cₓ of a native or iatrogenically administered substance X of the pulsating blood compartment if the concentration c_{Hb} of hemoglobin is known.

1. Noninvasive method for measuring blood components, wherein, with the use of spectrophotometry, light from at least one light source is generated and passed through a tissue located at an application site to at least one photoelectric transducer, and wherein at least one measuring signal of the photoelectric transducer is conducted to an evaluation unit, wherein light signals of a first wavelength are generated at two successive times T₁ and T₂, that light signals of a second wavelength are generated at two successive times T₃ and T₄, that light signals of a third wavelength are generated at two successive times T₅ and T₆, and that this procedure is continued for n pairs of times Tₙ and Tₙ₊₁ at n wavelengths. The times Tₙ . . . Tₙ₊₁ have a well-defined relationship with respect to time. Time differences between individual times can be small. The evaluation unit considers the incoming signals from the photoelectric transducer for all n wavelengths according to a predetermined computational model to determine the concentration of a blood component.

2. Method in accordance with claim 1, wherein the evaluation unit considers a quotient of the measuring signals.

3. Method in accordance with claim 1, wherein the logarithms of the measured values are taken.

4. Method in accordance with claim 1, wherein a quotient of the logarithmized measured values is considered.

5. Method in accordance with claim 1, wherein the light is generated by light-emitting diodes.

6. Method in accordance with claim 1, wherein the incoming signal is received by a photodiode.

7. Method in accordance with claim 1, wherein at least three different light sources are used.

8. Method in accordance with claim 1, wherein the total hemoglobin concentration is determined.

9. Method in accordance with claim 1, wherein the concentration of components that are not associated with hemoglobin is determined.

10. Method in accordance with claim 1, wherein the concentration of bilirubin is determined.

11. Method in accordance with claim 1, wherein the concentration of myoglobin is determined.

12. Method in accordance with claim 1, wherein the concentration of iatrogenically administered dyes is determined.

13. Device for measuring blood components, which has at least one light source, at least one photoelectric transducer, and at least one evaluation unit connected with the photoelectric transducer, wherein at least three light sources (1, 2, 3) are used, which generate wavelengths that are different from one another, and that the evaluation unit (6) has an arithmetic unit (7) both for taking logarithms and for performing divisions, multiplications, additions, and subtractions.

14. Device in accordance with claim 13, wherein at least one of the light sources (1, 2, 3) is realized as a light-emitting diode.

15. Device in accordance with claim 13, wherein the photoelectric transducer is realized as a photodiode.

16. Device in accordance with claim 13, wherein each of the light sources (1, 2, 3) generates light in a narrowly defined frequency band.

17. Device in accordance with claim 13, wherein one of the light sources (1, 2, 3) generates light with a wavelength of about 660 μm.

18. Device in accordance with claim 13, wherein one of the light sources (1, 2, 3) generates light with a wavelength of about 805 μm.

19. Device in accordance with claim 13, wherein one of the light sources (1, 2, 3) generates light with a wavelength of about 950 μm.
